(19) United States
(12) Patent Application Publication
(10) Pub. No.: US 2018/0296125 A1
(43) Pub. Date: Oct. 18, 2018

(54) METHODS, SYSTEMS, AND APPARATUS FOR DETECTING RESPIRATION PHASES

(71) Applicant: Intel Corporation, Santa Clara, CA (US)

(72) Inventors: Jie Zhu, San Jose, CA (US); Indira Negi, San Jose, CA (US)

(21) Appl. No.: 15/490,251

(22) Filed: Apr. 18, 2017

(51) Int. Cl. A61B 5/08; A61B 5/11; A61B 5/00

(52) U.S. Cl. CPC A61B 5/0816; A61B 5/11; A61B 5/7264; A61B 5/002; A61B 5/725; A61B 5/7203; A61B 5/6803

(57) ABSTRACT

Methods and apparatus for detecting respiration phases are disclosed herein. An example apparatus for analyzing vibration signal data collected from a nasal bridge of a subject via a sensor to reduce errors in training an artificial neural network using the vibration signal data includes a feature extractor to identify feature coefficients of the vibration signal data. In the example apparatus, the artificial neural network is to generate a respiration phase classification for the vibration signal data based on the feature coefficients. The example apparatus includes a classification verifier to verify the respiration phase classification and an output generator to generate a respiration phase output based on the verification.

### FIELD OF THE DISCLOSURE

This disclosure relates generally to respiration activity in subjects and, more particularly, to methods, systems, and apparatus for detecting respiration phases.

### BACKGROUND

Respiration activity in a subject includes inhalation and exhalation of air. Monitoring a subject's respiration activity can be used to obtain information for a variety of purposes, such as tracking exertion during exercise or diagnosing health conditions such as apnea. Breathing patterns derived from respiration data are highly subject-dependent based on physiological characteristics of the subject, the subject's health, etc. Factors such as environmental noise and subject movement can also affect the analysis of the respiration data and the detection of the respiration phases

### BRIEF DESCRIPTION OF THE DRAWINGS

FIG. 1 illustrates an example system including a nasal bridge vibration data collection device and a processing unit for detecting respiration phases constructed in accordance with the teachings disclosed herein.

FIG. 2 is a block diagram of an example implementation of a respiration phase detector of FIG. 1.

FIG. 3 is a block diagram of an example implementation of a post-processing engine of FIG. 2.

FIG. 4 illustrates a graph including example filtered signal data generated by example systems of FIGS. 1-3.

FIG. 5 illustrates a graph including a frame energy sequence generated by example systems of FIGS. 1-3.

FIG. 6 illustrates a graph including a segment of filtered signal data of FIG. 4.

FIG. 7 illustrates an example frequency spectrum generated based on the filtered signal data of FIG. 6.

FIG. 8 is a flowchart representative of example machine readable instructions that may be executed to implement the example systems of FIGS. 1-3.

FIG. 9 illustrates an example processor platform that may execute the example instructions of FIG. 8 to implement the example systems of FIGS. 1-3.

The figures are not to scale. Instead, to clarify multiple layers and regions, the thickness of the layers may be enlarged in the drawings. Wherever possible, the same reference numbers will be used throughout the drawing(s) and accompanying written description to refer to the same or like parts.

### DETAILED DESCRIPTION

Monitoring a subject's respiration activity includes collecting data during inhalation and exhalation by the subject. Respiration data can be collected from a subject via one or more sensors coupled to the subject to measure, for example, expansion and contraction of the subject's abdomen. In other examples, respiration data can be generated based on measurements of airflow volume through the subject's nose or acoustic breathing noises made by the subject. The respiration data can be analyzed with respect to breathing rate, duration of inhalations and/or exhalations, etc.

In examples disclosed herein, respiration data is derived from nasal bridge vibrations that are generated as the subject breathes. For example, the subject can wear a head-mounted device such as glasses that include one or more piezoelectric sensors coupled thereto. When the subject wears the glasses, the sensor(s) are disposed proximate to the bridge of the subject's nose. As the subject breathes (e.g., inhales and exhales), the piezoelectric sensor(s) deform and produce an electrical signal that can be analyzed to identify respiration patterns in the signal data.

Nasal bridge vibration data is highly individually dependent with respect to data patterns indicative of inhalation and exhalation. For example, strength and frequency of the nasal bridge vibration data varies by individual based on a manner in which the subject breathes, health conditions that may affect the subject's breathing rate, location(s) of the sensor(s) relative to the bridge of the subject's nose, a shape of the subject's nose, etc. Further, movement by the subject during data collection (e.g., head movements) adds noise to the signal data. Thus, characteristics of the nasal bridge vibration data generated by the sensor(s) can be inconsistent with respect to the subject during different data collection periods as well as between different subjects. Such variabilities in nasal bridge vibration data can affect reliability and accuracy in detecting respiration phases for the subject.

Example systems and methods disclosed herein analyze nasal bridge vibration data using a machine learning algorithm including a feedforward artificial neural network (ANN) to identify respiration phases including inhalation, exhalation, and non-breathing (e.g., noise). The ANN adaptively learns respiration phase classifications based on breathing interval patterns to classify characteristics or features of the nasal bridge vibration data. In some examples, the classified data is post-processed to verify the classification(s) by the ANN and/or to correct the classification(s) before outputting the identified respiration phases. In some examples, the results of the post-processing analysis are used to re-train the ANN with respect to identifying the respiration phases.

Some disclosed examples filter the nasal bridge vibration signal data to remove frequency components caused by movement(s) by the subject during data collection that may interfere with the accuracy of the analysis of the respiration data by the ANN. In some examples, peaks are identified in the filtered data and the locations of the peaks are used to identify substantially consistent breathing intervals (e.g., based on time between two inhalations or two exhalations). In some examples, the ANN is trained to classify the respiration phases when the breathing intervals are substantially consistent or below a breathing interval variance threshold. Thus, the ANN efficiently classifies the respiration phases based on data that does not include or is substantially free of anomalies such as a noise due to subject movements that could interfere with the application of learned classifications by the ANN.

Disclosed examples include a post-processing engine that evaluates the respiration phase classification(s) determined by the ANN and, in some examples, corrects the classification(s). The post-processing engine provides one or more outputs with respect to the identification of the respiration phases and average breathing rate. In some examples disclosed herein, the ANN adaptively learns or re-learns respiration phase features if the classification(s) are corrected during post-processing and/or if there are changes in the nasal bridge vibration data (e.g., due a change in respiration activity by the subject). Thus, disclosed examples address variability in nasal bridge vibration data through adaptive, self-learning capabilities of the ANN.

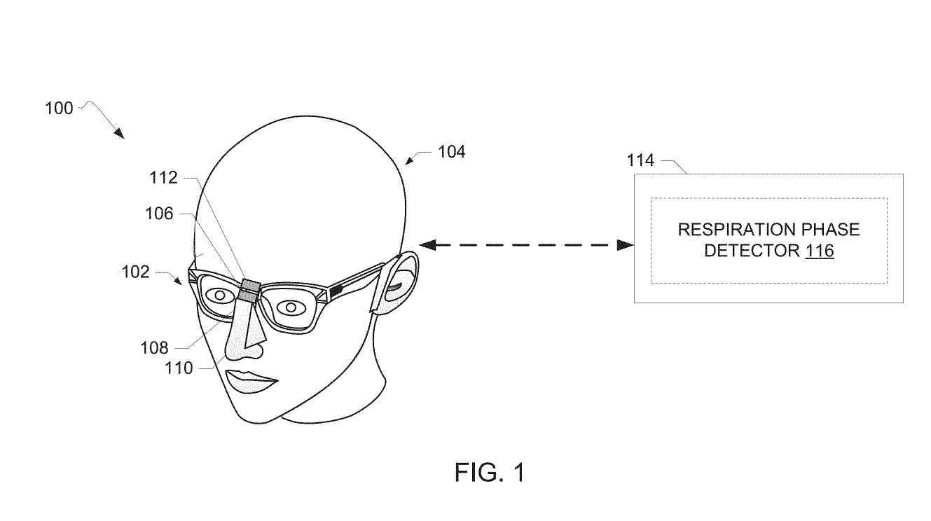

FIG. 1 illustrates an example system 100 constructed in accordance with the teachings of this disclosure for detecting respiration phases of a subject. The example system 100 includes a head-mounted device (HMD) 102 to be worn by a subject or user 104 (the terms “subject” and “user” may be used interchangeably herein). As illustrated in FIG. 1, the HMD 102 includes eyeglasses worn by the user 104. However, the HMD 102 can include other wearables, such as a mask or a nasal strip.

The HMD 102 includes one or more sensors 106 coupled to the HMD 102. In the example of FIG. 1, the sensor(s) 106 are piezoelectric sensor(s). The sensor(s) 106 are coupled to the HMD 102 such that when the user 104 wears the HMD 102, the sensor(s) 106 are disposed proximate to a bridge 108 of a nose 110 of the user 104. As the user 104 inhales and exhales, the sensor(s) 106 detect vibrations of the nasal bridge 108 due to the flow of air in and out of the user's nose 110. The sensor(s) 106 (e.g., piezoelectric sensor(s)) deform and generate electrical signal data based on the vibrations of the nasal bridge 108 during breathing. The sensor(s) 106 can measure the nasal bridge vibrations for a predetermined period of time (e.g., while the user 104 is wearing the HMD 102, for a specific duration, etc.).

The example HMD 102 of FIG. 1 includes a first processing unit 112 coupled thereto. The first processing unit 112 stores the vibration data generated by the sensor(s) 106. In some examples, the first processing unit 112 includes an amplifier to amplify the vibration data generated by the sensor(s) 106 and an analog-to-digital (A/D) converter to convert the analog signal data to digital data. In the example system 100 of FIG. 1, a second processing unit 114 is communicatively coupled to the first processing unit 112. The first processing unit 112 transmits (e.g., via Wi-Fi or Bluetooth connections or via cable connection) the vibration data to the second processing unit 114. The second processing unit 114 can be associated with, for example, a personal computer. In some examples, the data is transferred from the first processing unit 112 to the second processing unit 114 in substantially real-time as the data is being collected (e.g., in examples where the second processing unit 114 is disposed in proximity to the user 104 while the data is being collected). In other examples, the vibration data is transferred from the first processing unit 112 to the second processing unit 114 after a data collection period has ended.

The second processing unit 114 includes a respiration phase detector 116. The respiration phase detector 116 processes the vibration data obtained by the sensor(s) 106 to determine a breathing rate for the user 104. The respiration phase detector 116 identifies respiration phases (e.g., inhalation, exhalation) or non-breathing activity (e.g., noise) for the user 104 based on the vibration data. The respiration phase detector 116 can perform one or more operations on the vibration data such as filtering the raw signal data, removing noise from the raw signal data and/or analyzing the data. In some examples, one or more of the operations is performed by the first processing unit 112 (e.g., before the vibration data is transmitted to the second processing unit 114).

In some examples, the respiration phase detector 116 detects a change in the vibration data generated by the sensor(s) 106 and determines that the change is indicative of a change in a breathing pattern of the user 104. In such examples, the respiration phase detector 116 dynamically responds to the changes in the user's breathing pattern to identify the respiration phases based on characteristics or features of the current vibration data.

In some examples, the second processing unit 114 generates one or more instructions based on the determination of the breathing rate and/or the respiration phases to be implemented by, for example, the HMD 102. For example, the second processing unit 114 can generate a warning that the breathing rate of the user 104 is above a predetermined threshold and instruct the HMD 102 to present the warning (e.g., via a display of the HMD 102).

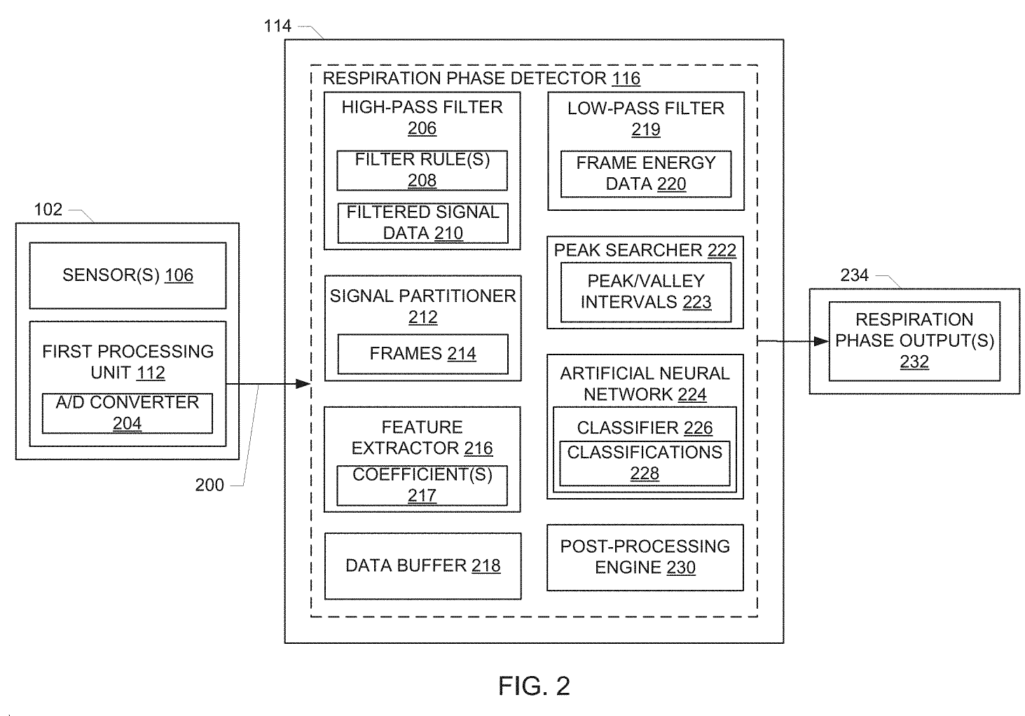

FIG. 2 is a block diagram of an example implementation of the example respiration phase detector 116 of FIG. 1. As mentioned above, the example respiration phase detector 116 is constructed to detect respiration phases (e.g., inhalation, exhalation) for a user based on nasal bridge vibration data generated by sensor(s) worn by the user (e.g., via a head-mounted device). In the example of FIG. 2, the respiration phase detector 116 is implemented by the example second processing unit 114 of FIG. 1. In other examples, the respiration phase detector 116 is implemented by the first processing unit 112 of the HMD 102 of FIG. 1. In some examples, one or more operations of the respiration phase detector 116 are implemented by the first processing unit 112 and one or more other operations are implemented by the second processing unit 114.

The example respiration phase detector 116 of FIG. 2 receives and/or otherwise retrieves nasal bridge vibration signal data 200 from the first processing unit 112 of the HMD 102. As disclosed above, the nasal bridge vibration signal data 200 is generated by the sensor(s) 106 while a user (e.g., the user 104 of FIG. 1) is wearing the HMD 102. The sensor(s) 106 measure vibrations of the nasal bridge of the user due to air flow during respiration. As illustrated in FIG. 2, in some examples, the first processing unit 112 includes an analog-to-digital (A/D) converter 204 to sample the vibration signal data 200 at a particular sampling rate (e.g., 2 kHz) and to covert the analog signal data to digital signal data for analysis by the example respiration phase detector 116.

The example respiration phase detection 116 of FIG. 2 includes a high-pass filter 206. The high-pass filter 206 can include, for example, a differentiator. The high-pass filter 206 of FIG. 2 filters the digital signal data generated by the A/D converter 204 to remove low frequency component(s) from the digital signal data. In the example of FIG. 2, the low frequency component(s) of the digital signal data may be associated with movements by the user that appear as noise in the vibration signal data 200. For example, during collection of the vibration signal data 200 by the sensor(s) 106, the user may voluntarily or involuntarily perform one or more movements that are detected by the sensor(s) 106, such as movements due to coughing and/or sneezing, facial movements, etc. In the example of FIG. 2, cutoff frequency ranges implemented by the high-pass filter 206 are based on one or more filter rule(s) 208. The filter rules 208 include predefined cutoff frequency ranges for known subject movements (e.g., head or facial movements). The filter rule(s) 208 may be received via one or more user inputs at the second processing unit 114. The high-pass filter 206 generates filtered digital signal data 210 as a result of the high-pass filtering.

The example respiration phase detector 116 includes a signal partitioner 212. The signal partitioner 212 partitions or divides the filtered signal data 210 into a plurality of portions or frames 214. The example signal partitioner 212 partitions the filtered signal data 210 based on time intervals. For example, the signal partitioner 212 partitions the filtered signal data 210 into respective frames 214 based on 100 milliseconds (ms) time intervals. In some examples, the frames 214 are divided based on 60 ms to 200 ms time intervals. In some examples, there is no overlap between the frames 214.

The example respiration phase detector 116 includes a feature extractor 216. The feature extractor 216 performs one or more signal processing operations on the frames 214 to characterize and/or recognize features in the signal data for each frame 214 that are indicative of respiration phases for the user. The feature extractor 216 characterizes the signal data by determining one or more feature coefficients 217 for each frame 214. For example, the feature extractor 216 performs one or more autocorrelation operations to calculate autocorrelation coefficient(s) including signal energy (e.g., up to an n^{th} order) for each frame 214. The feature coefficient(s) 217 determined by the feature extractor 216 can include the autocorrelation coefficients and/or coefficients computed from the autocorrelation coefficients, such as linear predictive coding coefficients or cepstral coefficients. In some examples, nine feature coefficients 217 are determined by the feature extractor 216. The feature extractor 216 can determine additional or fewer feature coefficients 217.

The feature coefficients 217 generated by the feature extractor 216 are stored in a data buffer 218 of the respiration phase detector 116. As disclosed herein, the features coefficients 217 stored in the data buffer 218 are used to train the respiration phase detector 116 to identify respiration phases in the frames 214. In the example of FIG. 2, the data buffer 218 is a first-in, first-out buffer.

The energy coefficient(s) determined by the feature extractor 216 for each frame 214 are filtered by a low-pass filter 219 of the example respiration phase detector 116 of FIG. 2. The cutoff frequency range used by the low-pass filter 219 of the respiration phase detector 116 is based on a particular breathing rate (e.g., 1 Hz-2 Hz). The low-pass filter 219 smooths frame energy data 220 (e.g., spectral energy data) for each of the frames 214.

The example respiration phase detector 116 includes a peak searcher 222. The peak searcher 222 analyzes the frame energy data 220 to determine whether the signal data is associated with a peak. The peak searcher 222 of FIG. 2 identifies the peaks based on the energy of the frames relative to a moving average of the frame energies filtered by the low-pass filter 219. For example, if a frame has a maximum energy among all consecutive frames whose number is not less than a preset positive integer and whose energy is greater than the moving average spanning a particular period of time (e.g., 10 seconds), then the peak searcher 222 identifies this frame with maximum energy as a peak.

Based on the identification of the peaks, the peak searcher 222 generates peak interval data 223 for alternating peak intervals. For example, where T(2k) is a time of a first peak (e.g., inhalation), T(2k−1) is a time of a second peak occurring one peak after the first peak (e.g., exhalation), T(2k−2) is a time of a third peak occurring two peaks after the first peak (e.g. inhalation), and T(2k−3) is a time of a fourth peak occurring three peaks after the first peak (e.g., exhalation), an interval between adjacent even peaks can be expressed as T(2k)−T(2k−2) and an interval between adjacent odd peaks can be expressed as T(2k−1)−T(2k−3). Thus, the peak searcher 222 identifies the locations of the peaks based on the energy coefficients derived from the filtered signal data 210. As disclosed herein, the locations of the peaks are used by the respiration phase detector 116 to verify the classification of the respiration phases.

The example respiration phase detector 116 of FIG. 2 includes a machine learning algorithm. In the example of FIG. 2, the machine learning algorithm is an artificial neural network (ANN) 224. The example ANN 224 of FIG. 2 is a feedforward ANN with one hidden layer. In the example of FIG. 2, the number of nodes at the input layer of the ANN 224 corresponds to the number of feature coefficients 217 calculated by the feature extractor 216. In the example of FIG. 2, the number of nodes at the output layer of the ANN 224 is two, corresponding to the identification of the respiration phases of inhalation and exhalation.

The example ANN 224 includes a classifier 226 to classify or assign the filtered signal data 210 of each frame 214 as either associated with outputs of [1, 0] or [0,1] corresponding to the respiration phases of inhalation or exhalation during training of the ANN 224. The classifier 226 classifies the signal data based on learned identifications of respiration feature patterns via training of the ANN 224. In some examples, the classifier 226 classifies the frames 214 over the duration that the vibration signal data 200 is collected from the user. In other examples, the classifier 226 classifies some of the frames 214 corresponding to the signal data collected from the user.

The classifier 226 generates classifications 228 with respect to the identification of the respiration phases in the signal data. For each frame 214, the classifier 226 outputs two numbers x, y between 0 and 1 (e.g., [x, y]). For example, if the classifier 226 identifies a frame 214 as including data having features indicative of inhalation, the classifier 226 should generate an output of [1,0] for the frame 214. If the classifier 226 identifies the frame 214 as including data having features indicative of exhalation, the classifier 226 should generate an output of [0, 1] for the frame 214. However, in operation, the [x, y] output(s) of the classifier 226 are not always [1, 0] or [0, 1].

The respiration phase detector 116 evaluates or post-processes the respiration phase classifications 228 by the classifier 226 to check for any error(s) in the classifications and correct the error(s) (e.g., by updating the classification with a corrected classification). The respiration phase detector 116 uses any corrections to the classifications 228 during post-processing to train or re-train the classifier 226 to identify the respiration phases. In some examples, the classifier 226 is re-trained in view of changes to the user's breathing pattern. In the example of FIG. 2, the respiration phase classifications 228 generated by the ANN 224 are analyzed by a post-processing engine 230 of the respiration phase detector 116.

The post-processing engine 230 receives the classifications 228 and the peak interval data 223 determined by the peak searcher 222 as inputs. The post-processing engine 230 evaluates the peak interval data 223 to determine whether the breathing intervals for the user are substantially consistent and, thus, to confirm that the signal data is sufficient for training the ANN 224 (e.g., the signal data is not indicative of non-normal breathing by the user). The post-processing engine 230 also evaluates the classifications 228 with respect to consistency of the classifications 228 by the ANN 224. For example, for three adjacent frames 214 each including signal data with energy above a predetermined threshold, the post-processing engine 230 verifies that the ANN 224 has correctly associated the frames with the same respiration phase (e.g., inhalation) and has not identified one of the frames as associated with the other respiration phase (e.g., exhalation). Thus, the post-processing engine 230 checks for errors in the classifications 228 by the ANN 224.

The post-processing engine 230 generates one or more respiration phase outputs 232. The respiration phase output(s) 232 can include locations of inhalation and exhalation phases in the signal data 210. The respiration phase output(s) 232 can include a breathing rate for the user based on the locations of the peaks. In some examples, the post-processing engine 230 generates one or more instructions for re-training the ANN 224 based on errors detected by the post-processing engine 230. The respiration phase output(s) 232 generated by the post-processing engine 230 can be presented via a presentation device 234 associated with the second processing unit 114 (e.g., a display screen). In some examples, the respiration phase output(s) 232 are presented via the first processing unit 112 of the head-mounted device 102.

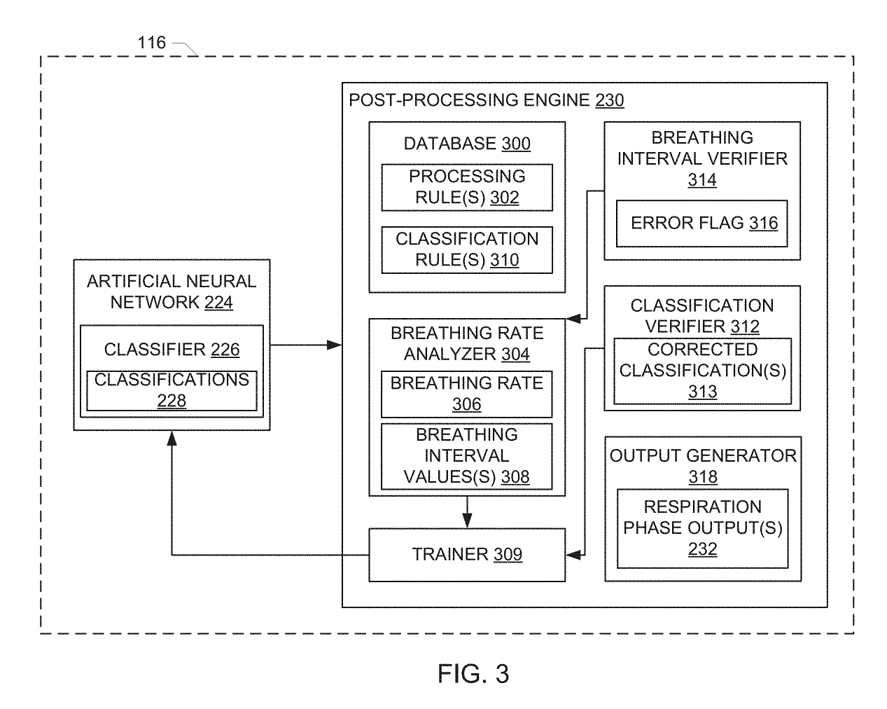

FIG. 3 is a block diagram of an example implementation of the example post-processing engine 230 of FIG. 2. For illustrative purposes, the example ANN 224 of the example respiration phase detector 116 of FIG. 2 is also illustrated in FIG. 3.

The post-processing engine 230 of FIG. 3 includes a database 300. The database 300 stores one or more processing rules 302. The processing rule(s) 302 include, for example, a maximum breathing interval variance for breathing patterns that are used to train the ANN 224, a predetermined error threshold for classifications by the ANN 224 to trigger re-training of the ANN 224, etc. The processing rule(s) 302 can be defined by one or more user inputs.

The example post-processing engine 230 includes a breathing rate analyzer 304. The breathing rate analyzer 304 uses the peak interval data 223 generated by the peak searcher 222 of the respiration phase detector 116 of FIG. 2 to estimate a breathing rate 306 for the user, or number of breaths per unit of time (e.g., 8 to 16 breaths per minute, where a breath includes inhalation and exhalation). For example, the breathing rate analyzer 304 can estimate the breathing rate 306 based on the number of peaks over a period of time. The breathing rate analyzer 304 of FIG. 3 calculates breathing interval value(s) 308 based on the reciprocal of the breathing rate 306. The breathing interval value(s) 308 represent a time between two inhalations or between two exhalations.

The breathing rate analyzer 304 compares two or more of the breathing interval values 308 with respect to a variance between the breathing intervals to determine when the breathing interval for the user is substantially consistent. For example, a consistent breathing interval D(k) including inhalation and exhalation can be represented by the expression:

T(2k)−T(2k−2)=T(2k−1)−T(2k−3)=D(k), where T represents time and k represents a peak location or index, such that T(2k) is a time of a first peak (e.g., inhalation), T(2k−1) is a time of a second peak occurring one peak after the first peak (e.g., exhalation), T(2k−2) is a time of a third peak occurring two peaks after the first peak (e.g. inhalation), and T(2k−3) is a time of a fourth peak occurring three peaks after the first peak (e.g., exhalation) (Equation 1).

However, due to noise and/or slight variations in the user's breathing, there may be some variance with respect to the times between the user's inhalations or exhalations. In some examples, the breathing rate analyzer 304 determines when a variance between the breathing interval values 308 is at or below a particular breathing interval variance threshold such that the breathing interval is substantially consistent. The particular variance threshold can be based on the processing rule(s) 302 stored in the database 300.

When the breathing rate analyzer 304 determines that the breathing interval is substantially consistent, the breathing rate analyzer 304 determines that the user's breathing is substantially regular (e.g., normal) for the user and, thus, the signal data 210 is adequate for training the ANN 224. Irregular breathing patterns due to, for example, illness, are not reflective of the user's typical breathing pattern. Thus, identifying respiration phases based on data associated with inconsistent breathing intervals would be inefficient with respect to training the ANN 224 to recognize user-specific respiration phases because of the variability in the signal data.

The example post-processing engine 230 includes a trainer 309. The trainer 309 trains the ANN 224 to classify the signal data in each of the frames 214 based on one or more classification rules 310 stored in the database 300 of FIG. 3. As disclosed herein, the classification rules 310 are also used by the post-processing engine 230 to verify that the classifier 226 has correctly identified the respiration phases for the frames 214. In some examples, the trainer 309 uses the data (e.g., the feature coefficients 217) stored in the data buffer 218 of FIG. 2 to train the ANN 224. In some examples, the post-processing engine 230 sets a ANN training flag to indicate that the ANN 224 should be trained (e.g., via the trainer 309).

For example, the classification rules 310 can indicate that peaks labeled inhalation and exhalation should alternate (e.g., based on a user breathing in-out-in-out). The classification rules 310 can include a rule that a peak is limited by two adjacent valleys. The classification rules 310 can include a rule for training the ANN 224 that if a first peak has a longer duration than a second peak, then the first peak should be labeled as exhalation. The classification rules 310 can include an energy threshold for identifying the data as associated with inhalation or exhalation (e.g., based on the energy coefficients). The energy threshold may be a fraction of the moving average of previous frame energies. The classification rules 310 can include a rule that if the classifier 226 identifies the data in a frame 214 as associated with inhalation, the classifier 226 should output a classification 228 of [1, 0]. The classification rules 310 can include a rule that if the classifier 226 identifies the data in a frame 214 as associated with exhalation, the classifier 226 should output a classification 228 of [0,1].

In some examples, an inhalation phase in the signal data 210 may have a longer duration than an individual frame 214. Thus, the inhalation phase may extend over a plurality of frames 214. Similarly, an exhalation phase in the signal data 210 may have a longer duration than an individual frame 214. Thus, the exhalation phase may extend over a plurality of frames 214. The example classification rule(s) 310 include a rule that consecutive frames 214 including signal data with energy over a particular threshold should be classified as the same phase.

Based on the training by the example trainer 309 of FIG. 3, the classifier 226 of the ANN 224 classifies the data in the respective frames 214 with respect to a respiration phase. As disclosed above, the classifier 226 analyzes the input features coefficients 217 and generates two numbers [x, y] (where x and y are between 0 and 1) for each frame 214 indicating whether the data is associated with inhalation or exhalation. In some examples, the classifier 226 analyzes the [x, y] outputs for a plurality of frames 214 having similar energy coefficients (e.g., corresponding to a peak) to determine whether the respiration phase for the signal data from which the frames 214 are generated is inhalation or exhalation.

In the example of FIGS. 2 and 3, although the classifier 226 of the ANN 224 is trained to output [1, 0] for the inhalation phase and [0, 1] for the exhalation phase, in some examples, the classifier 226 outputs x and/or y values between 0 and 1 for one or more frames 214 due to, for example, noise in the data. For example, for consecutive first, second, and third frames 214, the classifier 226 may output values of [1, 0] for the first frame, [0.8, 0.2] for the second frame, and [0.9, 0.1] for the third frame. In such examples, a classification verifier 312 of the post-processing engine 230 determines that the mean of the x values for the frames (i.e., 0.9 in this example) is greater than θ, where θ is in the interval [0.5, 1] (e.g. θ=0.7)and, in particular, closer to the value of 1. The classification verifier 312 determines that the mean of they values for the frames (i.e., 0.1 in this example) is less than 1−θ, and, in particular, is closer to 0. Based on the mean of the x values being closer to 1 and the mean of they values being closer to 0, the classification verifier 312 of the post-processing engine 230 identifies the signal data for the frames as associated with the inhalation phase (e.g., based on the classification rule(s) 310 indicating that an output of [1, 0] is representative the inhalation phase). In other examples, the classification verifier 312 determines that the signal data of the frames is associated with the exhalation phase if the mean of they values is closer to 1>θ and the mean of the x values is less than 1−θ, per the example classification rule 310 indicating that the numbers [0, 1] are associated with the exhalation phase. In some examples, if either of the mean of the x values or the mean of they values is in the interval [1−θ, θ] for a particular threshold θ, then the signal data is considered indicative of non-breathing activity or untrained breathing activity (e.g., breathing data for which the ANN 224 has not been trained).

Thus, the classifier 226 of the ANN 224 classifies the respiration phases based on the signal data in each frame 214 (e.g., based on the feature coefficients 217 such as the energy coefficients) and the training of the ANN 224 in view of the classification rules 310. However, in some examples, despite the training of the ANN 224, the classifier 226 incorrectly classifies the signal data of one or more of the frames 214. For example, classification errors may arise from the fact that the user may not breathe exactly the same way every time data is collected. Classification errors may also arise from anomalies in the user's data, such as a sudden change in duration between inhalations or exhalations in an otherwise substantially consistent breathing interval.

The example classification verifier 312 of the post-processing engine 230 includes detects and corrects errors in the classifications 228 by the classifier 226 of the ANN 224. For example, to detect classification errors, the classification verifier 312 evaluates the [x, y] outputs for a plurality of the frames 214 relative to one another. As disclosed above, data corresponding to a respiration phase can extend over two or more frames 214. For example, a peak associated with an inhalation phase can extend over ten consecutive frames (e.g., a first frame, a second frame, a third frame, etc.). The classifier 226 may output the numbers [1, 0] for the first frame; [0, 1] for the second frame, and [1, 0] for the remaining frames. As disclosed above, the classifier 226 is trained to output the number [1, 0] for inhalation. Thus, the classifier 226 determined that the signal data of all except for the second frame is associated with the inhalation phase. The classification verifier 312 detects that the classification for the second frame (i.e., [0, 1]) is associated with the exhalation phase. The classification verifier 312 also recognizes that the second frame is disposed between the first frame and the third frame, both of which were classified as associated with the inhalation phase. The classification verifier 312 can analyze the energy of the signal data in the second frame and determine that the energy is similar to the energy of the first and third frames. As a result, the classification verifier 312 determines that the phase assignment for the second frame is incorrect. The classification verifier 312 corrects the classification of the data of the second frame (e.g., by updating the classification with a corrected classification 313) so that the outputs for the first, second, and all remaining frames correspond to the inhalation phases. The classification verifier 312 generates the corrected classification 313 for the second frame based on, for example, the classification rule(s) 310 indicating that adjacent frames with similar characteristics (e.g., energy levels) are associated with the same respiration phase.

Based on the errors detected in classification outputs by the classifier 226, the classification verifier 312 may determine that the ANN 224 needs to be re-trained with respect to identifying the respiration phases. In the example of FIG. 3, the classification verifier 312 determines that the ANN 224 needs to be re-trained if either the mean of the x values or the mean of the y values of the ANN classifier outputs [x, y] is in the interval [1−Ω. Ω] for a particular re-training threshold Ω (e.g., Ω>θ). Put another way, the classification verifier 312 determines that the ANN 224 needs to be re-trained if the mean x of the x values is x≤Ω or the mean y of they values is y>1−Ω for an expected output of [1, 0] or, x≥Ω or y<1−Ω for an expected output of [0, 1]. The classification verifier 312 communicates with the trainer 309 to re-train the ANN 224. In some examples, the trainer 309 re-trains the ANN 224 based on the signal data associated with the respiration phase which the classifier 226 incorrectly identified and the data for previously identified phases (e.g., associated with immediately preceding frames). In some examples, the trainer 309 uses data stored in the data buffer 218 of FIG. 2 during the re-training, such as the feature coefficients identified for the signal data used to re-train the ANN 224.

In some examples, the classification verifier 312 determines that ANN 224 was unable to classify the signal data 210. For example, the classification verifier 312 may detect classification errors above a particular error threshold (e.g., as defined by the processing rule(s) 302). In such examples, the post-processing engine 230 checks the breathing interval values 308 of the signal data to verify that the breathing interval values 308 meet a breathing interval variance threshold and, thus, the breathing interval is substantially consistent. In the example of FIGS. 2 and 3, if the breathing interval is not substantially consistent, the trainer 309 does not re-train the ANN 224.

The example post-processing engine 230 of FIG. 3 includes a breathing interval verifier 314. As disclosed above, a consistent breathing interval including inhalation and exhalation can be represented by Equation 1 above (i.e., T(2k)−T(2k−2)=T(2k−1)−T(2k−3)=D(k) for a specific index k). However, in some examples, the breathing intervals D(k) are not equal due to estimation errors of peak locations and breathing pattern variance. . In such examples, a smoothing breathing interval D(n) is used and updated such that for every n:

D(n+1)=(1−μ)*D(n)+μ*(T(n+2)−T(n)), where n is a current sample index and where μ is a particular positive number less than 1 and indicative of a smoothing factor to reduce of the estimation errors of peak locations and breathing pattern variance (Equation 2).

In some examples, the breathing interval verifier 314 determines that, despite the removal of the noise, the limitation (T(n+2)−T(n)) in Equation 2, above, is not within a particular (e.g., predefined) threshold range. For example, if T(n+2)−T(n)−D(n) is greater than a particular (e.g., predefined) breathing interval variance threshold (e.g., as defined by the processing rule(s) 302), then the breathing interval verifier 314 sets an error flag 316. The error flag 316 indicates that the breathing interval is not substantially consistent and, thus, the ANN 224 should not be re-trained. In such examples, the breathing interval verifier 314 instructs the breathing rate analyzer 304 to monitor the peak interval data 223 to identify when the breathing interval is substantially consistent and, thus, the signal data is adequate to be used to re-train the ANN 224.

In the example of FIG. 3, if the error flag 316 is set by the breathing interval verifier 314, then the data associated with the error flag is not used to re-train the ANN 224. As disclosed above, using data indicative of inconsistent breathing patterns to train the ANN 224 is inefficient with respect teaching the ANN 224 to identify respiration phases because of the variability in the data. Also, noise patterns are not used to train the ANN 224 because it may be difficult for the ANN 224 to distinguish between noise and respiration due to the variability in noise signals.

The example post-processing engine 230 includes an output generator 318. The output generator 318 generates the respiration phase output(s) 232 based on the review of the classifications 228 by the ANN 224. For example, the output generator 318 generates the outputs 232 with respect to the locations of the inhalation and exhalation phases in the signal data 210. In some examples, the output(s) 232 include corrected classifications made by the classification verifier 312 if the classification verifier 312 detects errors in the classifications by the ANN 224. In some examples, the output(s) 232 include a breathing rate for the user (e.g., the inverse of the breathing interval or 1/D(n)).

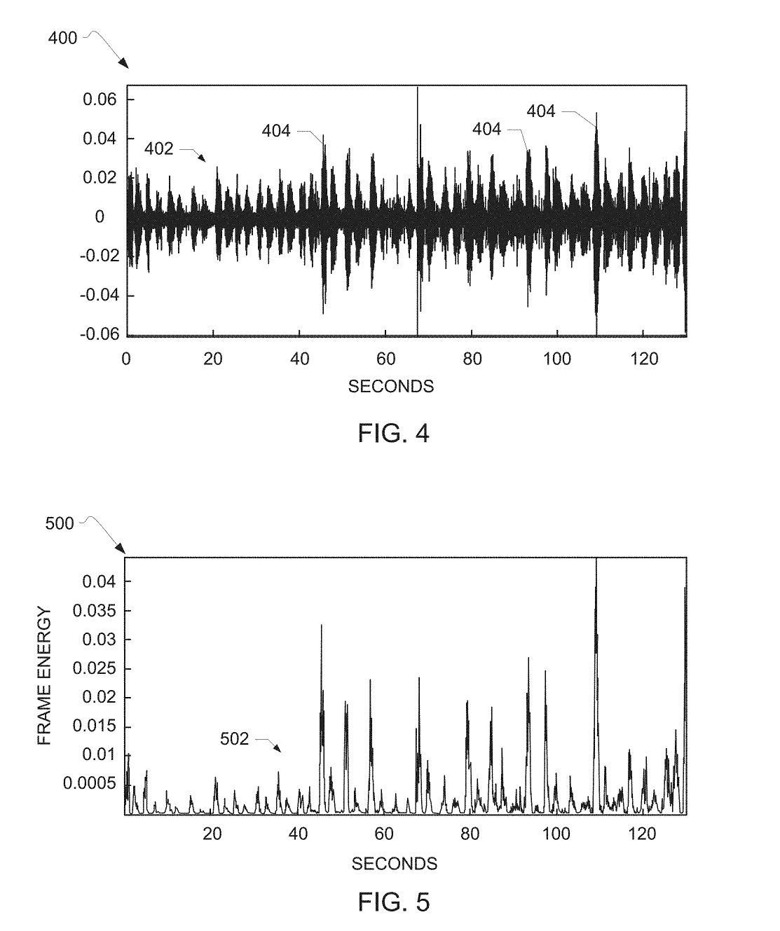

FIG. 4 illustrates an example graph 400 including filtered signal data 402 generated by, for example, the example high-pass filter 206 of the respiration phase detector 116 of FIGS. 2 and 3. As illustrated in FIG. 4, the filtered signal data 402 is generated based on nasal bridge vibration data (e.g., the vibration signal data 200 of FIG. 2) collected from a user (e.g., the user 104) over approximately a 120 second time period. The filtered signal data 402 includes breathing-activity data 404 indicative of inhalation or exhalation by the user.

FIG. 5 illustrates an example graph 500 including a frame energy sequence 502 for frames (e.g., the frames 214) generated from the filtered signal data 402 of the example graph of FIG. 4. The example frame energy sequence 502 can be generated by the feature extractor 216 of the example respiration phase detector 116 of FIG. 2 based on energy coefficients (e.g., the feature coefficients 217) determined for each frame. The example frame energy sequence 502 of FIG. 5 can be filtered by the example low-pass filter 219 of FIG. 2 and used by the example peak searcher 222 of FIG. 2 to generate the peak interval data 223.

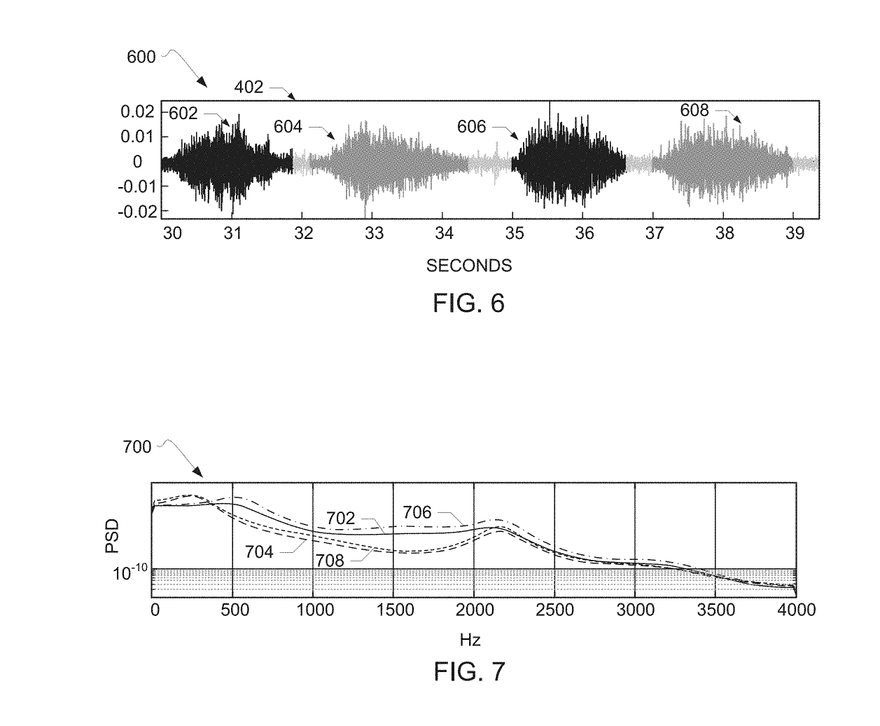

FIG. 6 illustrates an example graph 600 including a segment of the example filtered signal data 402 of the example graph 400 of FIG. 4 for the time period between 30-39 seconds. As shown in FIG. 6, the filtered signal data includes first breathing activity data 602, second breathing activity data 604, third breathing activity data 606, and fourth breathing activity data 608. As disclosed above, a user typically breathes by alternating inhalations and exhalations. In the example of FIG. 6, the first breathing activity data 602 and the third breathing activity data 606 are associated with a first respiration phase (e.g., inhalation) and the second breathing activity data 604 and the fourth breathing activity data 608 are associated with a second respiration phase (e.g., exhalation). The example breathing activity data 602, 604, 606, 608 can also be used by the example breathing rate analyzer 304 of FIG. 3 to determine if the breathing interval is substantially consistent based on, for example, durations between adjacent inhalations and exhalations relative to a breathing interval variance threshold.

FIG. 7 is an example frequency spectrum 700 for the first breathing activity data 602, second breathing activity data 604, third breathing activity data 606, and fourth breathing activity data 608 of FIG. 6. The example frequency spectrum 700 can be generated by the example respiration phase detector 116 of FIG. 2 based on the feature coefficients 217 determined by the autocorrelation operations for the signal data 602, 604, 606, 608. The example of frequency spectrum 700 includes first spectral data 702 based on the first breathing activity data 602, second spectral data 704 based on the second breathing activity data 604, third spectral data 706 based on the third breathing activity data 606, and fourth spectral data 708 based on the fourth breathing activity data 608.

As illustrated in FIG. 7, a shape of the first spectral data 702 and a shape of the third spectral data 706 are substantially similar, reflecting the association of the first breathing activity data 602 and the third breathing activity data 606 with the same respiration phase. As also illustrated in FIG. 7, a shape of the second spectral data 704 and a shape of the fourth spectral data 708 are substantially similar, reflecting the association of the second breathing activity data 604 and the fourth breathing activity data 608 with the same respiration phase. The example ANN 224 of FIGS. 2 and 3 is trained to output the same respiration phase classifications 228 for the first breathing activity data 602 and the third breathing activity data 606 (e.g., [1, 0] for inhalation) and the same respiration phase classifications 228 for the second breathing activity data 604 and the fourth breathing activity data 608 (e.g., [0, 1] for exhalation). The ANN 224 classifies the spectral data for each frame by generating an output of, for example, [1, 0] for the inhalation phase and [0, 1] for the exhalation phase based on the analysis of the spectral data. As disclosed above, the post-processing engine 230 can verify the classifications 228 by comparing the classifications for consecutive frames to confirm that the classifications are consistent. For example, the classification verifier 312 of FIG. 3 can verify that the outputs generated based on the first breathing activity data 602 are associated with the inhalation phase (e.g., x of [x, y] is close to 1 and y of [x, y] is close to 0).

While an example manner of implementing the example respiration phase detector 116 are illustrated in FIGS. 1-3, one or more of the elements, processes and/or devices illustrated in FIGS. 1-3 may be combined, divided, re-arranged, omitted, eliminated and/or implemented in any other way. Further, the example A/D converter 204, the example high-pass filter 206, the example signal practitioner 212, the example feature extractor 216, the example data buffer 218, the example low-pass filter 219, the example peak searcher 222, the example ANN 224, the example classifier 226, the example post-processing engine 230, the example database 300, the example breathing rate analyzer 304, the example trainer 309, the example classification verifier 312, the example breathing interval verifier 314, the example output generator 318 and/or, more generally, the example respiration phase detector 116 of FIGS. 1-3 may be implemented by hardware, software, firmware and/or any combination of hardware, software and/or firmware. Thus, for example, any of the example A/D converter 204, the example high-pass filter 206, the example signal practitioner 212, the example feature extractor 216, the example data buffer 218, the example low-pass filter 219, the example peak searcher 222, the example ANN 224, the example classifier 226, the example post-processing engine 230, the example database 300, the example breathing rate analyzer 304, the example trainer 309, the example classification verifier 312, the example breathing interval verifier 314, the example output generator 318 and/or, more generally, the example respiration phase detector 116 of FIGS. 1-3 could be implemented by one or more analog or digital circuit(s), logic circuits, programmable processor(s), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example the example A/D converter 204, the example high-pass filter 206, the example signal practitioner 212, the example feature extractor 216, the example data buffer 218, the example low-pass filter 219, the example peak searcher 222, the example ANN 224, the example classifier 226, the example post-processing engine 230, the example breathing rate analyzer 304, the example trainer 309, the example classification verifier 312, the example breathing interval verifier 314, the example output generator 318 and/or, more generally, the example respiration phase detector 116 is/are hereby expressly defined to include a non-transitory computer readable storage device or storage disk such as a memory, a digital versatile disk (DVD), a compact disk (CD), a Blu-ray disk, etc. storing the software and/or firmware. Further still, the example respiration phase detector 116 of FIGS. 1-3 may include one or more elements, processes and/or devices in addition to, or instead of, those illustrated in FIG. 3, and/or may include more than one of any or all of the illustrated elements, processes and devices.

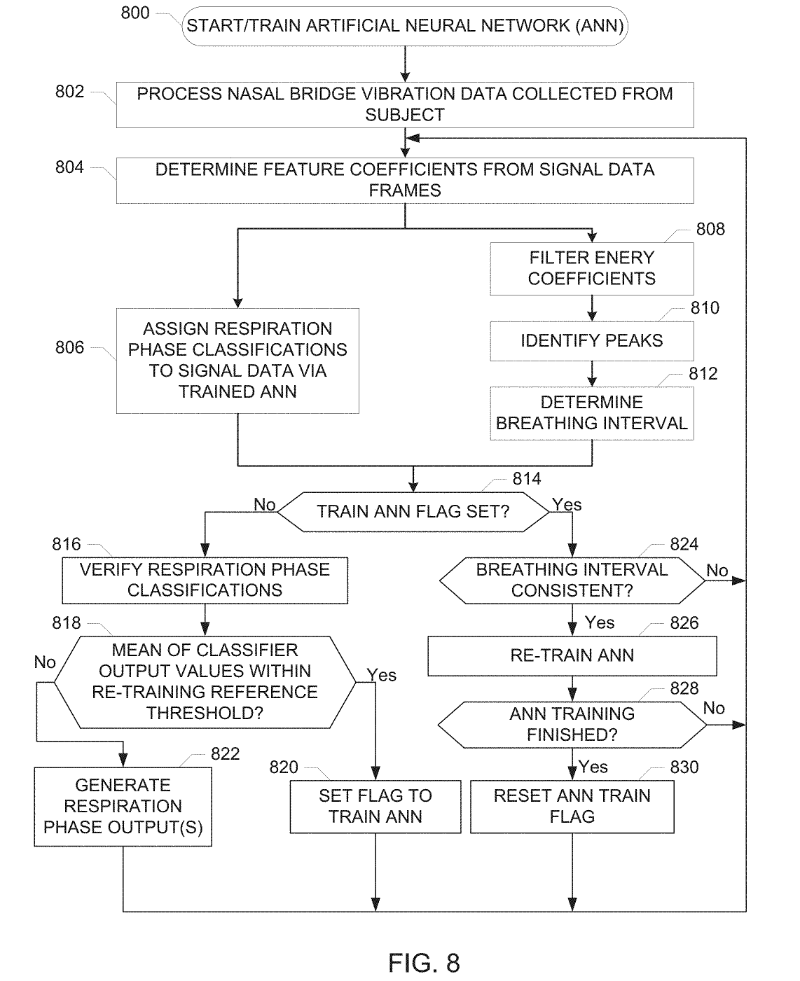

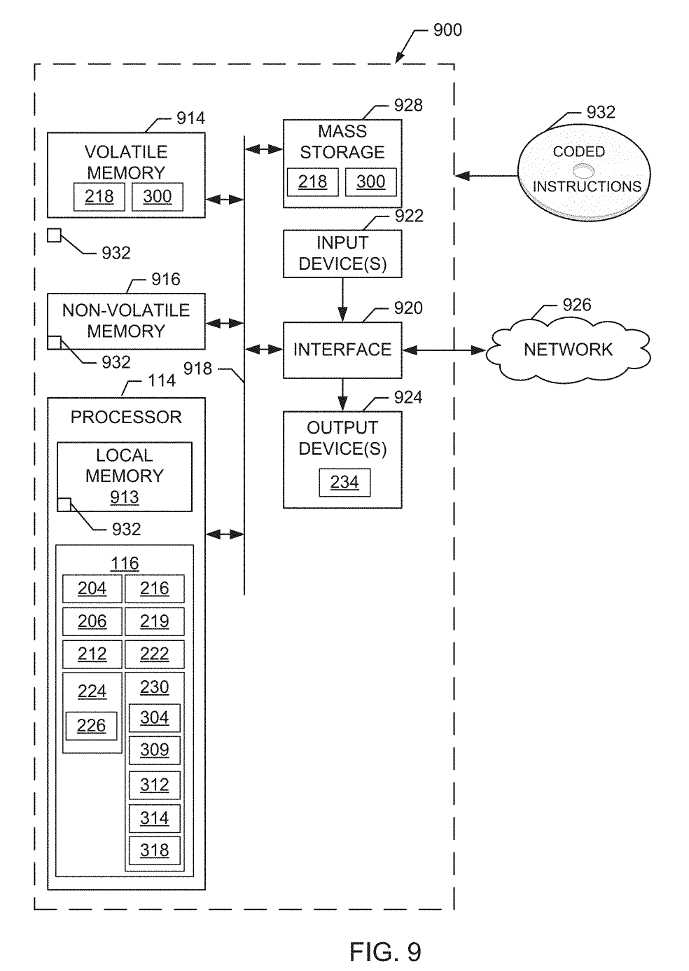

A flowchart representative of example machine readable instructions for implementing the example system 100 of FIGS. 1-3 is shown in FIG. 8. In this example, the machine readable instructions comprise a program for execution by one or more processors such as the processor 114 shown in the example processor platform 900 discussed below in connection with FIG. 9. The program may be embodied in software stored on a non-transitory computer readable storage medium such as a CD-ROM, a floppy disk, a hard drive, a digital versatile disk (DVD), a Blu-ray disk, or a memory associated with the processor 114, but the entire program and/or parts thereof could alternatively be executed by a device other than the processor 114 and/or embodied in firmware or dedicated hardware. Further, although the example program is described with reference to the flowchart illustrated in FIG. 8, many other methods of implementing the example system 100 and/or components thereof may alternatively be used. For example, the order of execution of the blocks may be changed, and/or some of the blocks described may be changed, eliminated, or combined.

As mentioned above, the example process of FIG. 8 may be implemented using coded instructions (e.g., computer and/or machine readable instructions) stored on a non-transitory computer readable storage medium such as a hard disk drive, a flash memory, a read-only memory (ROM), a compact disk (CD), a digital versatile disk (DVD), a cache, a random-access memory (RAM) and/or any other storage device or storage disk in which information is stored for any duration (e.g., for extended time periods, permanently, for brief instances, for temporarily buffering, and/or for caching of the information). As used herein, the term non-transitory computer readable storage medium is expressly defined to include any type of computer readable storage device and/or storage disk and to exclude propagating signals and to exclude transmission media. As used herein, “non-transitory computer readable storage medium” and “non-transitory machine readable storage medium” are used interchangeably. As used herein, when the phrase “at least” is used as the transition term in a preamble of a claim, it is open-ended in the same manner as the term “comprising” is open ended.

FIG. 8 is a flowchart of example machine-readable instructions that, when executed, cause the example respiration phase detector 116 of FIGS. 1,2, and/or 3 to detect respiration phases based on nasal bridge vibration data collected from a subject (e.g., the user 104 of FIG. 1). In the example of FIG. 8, the nasal bridge vibration data can be generated by a subject wearing a head-mounted device (e.g., the HMD 102 of FIGS. 1 and 2) including sensor(s) (e.g., the sensor(s) 106) to generate the vibration data. The example instructions of FIG. 8 can be executed by the second processing unit 114 of FIGS. 1-3. One or more of the instructions of FIG. 8 can be executed by the first processing unit 112 of the HMD 102 of FIGS. 1 and 2.

The example of FIG. 8 uses the previously trained artificial neural network (ANN) 224 of FIGS. 2-3 to detect respiration phases in the nasal bridge vibration data 200 collected from a subject (block 800). The ANN 224 is trained by the trainer 309 of FIG. 3 to recognize the respiration phases in the signal data based on the feature coefficients 217 (e.g., including signal energy), which serve as inputs to the ANN 224, and one or more classification rule(s) 310 for classifying the data (e.g., based on particular (e.g., predetermined) energy thresholds, rules regarding the classifications of consecutive frames, etc.). In the example of FIG. 8, the ANN 224 is trained using signal data indicative of a substantially consistent breathing interval for the subject based on a breathing interval variance threshold (e.g., substantially consistent intervals between inhalations or exhalations).

In the example of FIG. 8, the example respiration phase detector 116 of FIGS. 2-3 processes the nasal bridge vibration data 200 collected from the subject using the sensor(s) 106 and received at the second processing unit 114 via, for example the first processing unit 112 of the HMD 102 (block 802). For example, the A/D converter 204 of the example first processing unit 112 of FIGS. 1-2 converts the raw vibration signal data 200 to digital signal data. The high-pass filter 206 of the example respiration phase detector 116 of FIG. 2 filters the digital signal data to remove, for example, low frequency components in the data due to movements by the subject based on one or more filter rule(s) 208. The high-pass filter 206 generates the filtered signal data 210. The example signal partitioner 212 partitions the filtered signal data 210 into a plurality of frames 214 based, for example, particular (e.g., 100 ms) time intervals.

The feature extractor 216 of the example respiration phase detector 116 of FIGS. 2-3 determines the feature coefficients 217 (e.g., including signal energy) from the filtered signal data 210 for each of the frames 214 (block 804). The example feature extractor 216 uses one or more signal processing operations (e.g., autocorrelation) to determine the coefficients 217. In some examples, the coefficients are stored in the data buffer 218 to train the ANN 224.

In the example of FIG. 8, the feature coefficients 217 are provided as inputs to the ANN 224. The classifier 226 of the example ANN 224 of FIGS. 2 and 3 assigns respiration phase classifications to the signal data based on the training of the ANN 224 (block 806). The classifier 226 generates classifications 228 for the frames 214 assigns the classifications 228 the signal data in the frames 214 as associated with inhalation, exhalation, or non-breathing activity (e.g., noise). In some examples, the classifier 226 outputs two numbers between 0 and 1 (e.g., [x, y]) as the classification 228 for a frame 214. In some such examples, the classification verifier 312 of the post-processing engine 230 determines respective means of the x and y values assigned to two or more consecutive frames 214 to classify breathing activity including a peak (e.g., a the breathing activity having a length that spans the frames) as associated with inhalation or exhalation by comparing the respective means of the x and y values to a particular threshold θ (e.g., classifier verifier 312 determines a frame is associated with inhalation if a mean x of the x values is greater than θ (and, in particular is closer to a value of 1) and a mean y of they values is less than 1−θ (and, in particular is closer to a value of 0)).

Also, in the example of FIG. 8, the energy coefficients of the frames 214 determined by the feature extractor 216 of FIG. 2 are low-passed filtered by the example low-pass filter 219 of FIG. 2 (block 808). The low-pass filter 219 generates the frame energy data 220 (e.g., spectral energy data) based on the filtering.

In the example of FIG. 8, the peak searcher 222 analyzes the frame energy data 220 to identify peaks in the signal data 210 (block 810). The peak searcher 222 generates the peak interval data 223 including the locations of the peaks in the signal data 210.

In the example of FIG. 8, the breathing rate analyzer 304 of the example post-processing engine 230 of FIGS. 2 and 3 analyzes the peak interval data 223 to determine the breathing rate 306 and the breathing interval value(s) 308 for the subject (block 812). For example, the breathing rate analyzer 304 can determine the breathing interval value(s) 308 (e.g., the time between two adjacent inhalations or two adjacent exhalations) based on the inverse of the breathing rate 306, or the number of breaths per minute.

The example of FIG. 8 includes a determination of whether a flag is set to train the ANN 224 with respect to classifying the signal data (block 814). The training flag can be set by, for example, the post-processing engine 230 (e.g. the trainer 309).

In the example of FIG. 8, the classification(s) 228 generated by the classifier 226 of the example ANN 224 of FIGS. 2 and 3 are verified by the example post-processing engine 230 of FIGS. 2 and 3 (block 816). For example, the classification verifier 312 of the post-processing engine 230 verifies the classification(s) 228 based on the processing rule(s) 302 and/or the classification rule(s) 310 stored in the database 300 of the post-processing engine 230 of FIGS. 2 and 3. The classification verifier 312 identifies any errors in the classification outputs for the frames 214, such as an output indicative of exhalation (e.g., [0, 1]) for data of a frame located between two frames include data classified as associated with inhalation (e.g., [1,0]). In some examples, the classification verifier 312 corrects the classification(s) (e.g., by updating the classification(s) 228 with corrected classification(s) 313) if error(s) are detected.

In the example of FIG. 8, the classification verifier 312 analyzes the means of each of the values (e.g., the x and y values) output by the ANN classifier 328 relative to a re-training reference threshold Q (block 818). In the example of FIG. 8, the classification verifier 312 determines that the ANN 224 needs to be re-trained if either the mean of the x values or the mean of they values of the ANN classifier outputs [x, y] is in the interval [1−Ω. Ω] for the particular re-training threshold Ω (e.g., Ω>θ).

In the example of FIG. 8, if the classification verifier 312 determines that either the mean of the x values or the mean of they values of the ANN classifier outputs [x, y] is in the interval [1−Ω. Ω], then the classification verifier determines that the re-training threshold has been met and the ANN 224 needs to be retrained. If the classification verifier 312 determines that the ANN 224 needs to be re-trained, the trainer 309 of the example post-processing engine 230 sets the flag to indicate that the ANN 224 needs to be re-trained (block 820).

In the example of FIG. 8, if the classification verifier 312 determines that the mean of the x values or the mean of they values is not in the interval [1−Ω. Ω], then the output generator 318 generates the respiration phase output(s) 232 (block 822). The respiration phase output(s) 232 can be displayed via, for example, a presentation device 234 associated with the second processing unit 114 or, in some examples, the HMD 102. The respiration phase output(s) 232 can include the location of the inhalation and exhalation respiration phases in the signal data and/or a breathing rate for the subject. In some examples, the identification of the inhalation and exhalation respiration phases is based on corrections to the classifications 228 by the classification verifier 312 if errors were detected.

In the example of FIG. 8, if the ANN training flag is set (block 814), and if the breathing interval verifier 314 confirms that the signal data includes a substantially consistent breathing interval (block 824), the ANN 224 is trained via the trainer 309 of the post-processing engine 230 (block 826). The breathing interval verifier 314 determines that the breathing interval is substantially consistent if the breathing interval values meet a particular breathing interval variance threshold. If the breathing interval verifier 314 determines that the breathing interval is not substantially consistent, the example post-processing engine 230 does not use the breathing interval data to re-train the ANN 224. The example breathing rate analyzer 304 monitors the signal data to identify when the data reflects a substantially consistent breathing interval that is adequate for (re-)training of the ANN 224 and returns to train the ANN 224 when a substantially consistent breathing interval is identified.

In the example of FIG. 8, the trainer 309 of the post-processing engine 230 re-trains the ANN 224 to identify the respiration phases using, for example, data for the frame which was incorrectly classified and data for previous frames that were correctly classified (e.g., immediately preceding frames). In some examples, the trainer 309 uses the feature coefficients 217 for the frames stored in the data buffer 218 of FIG. 2 to re-train the ANN 224.

The example of FIG. 8 continues to train the ANN 224 until a determination that the training of the ANN 224 is finished (block 828). If the training of the ANN is finished, the trainer 309 resets the ANN training flag (block 830). The example of FIG. 8 continues to monitor the nasal bridge vibration data received by the respiration phase detector 116 if FIGS. 1-3. The example instructions of FIG. 8 may be re-implemented reiterated when complete and/or as needed to train the ANN 224 and identify respiration phases in nasal bridge vibration data.

FIG. 9 is a block diagram of an example processor platform 900 capable of executing the instructions of FIG. 8 to implement the example respiration phase detector 116 of FIGS. 1, 2, and/or 3. The processor platform 900 can be, for example, a server, a personal computer, a mobile device (e.g., a cell phone, a smart phone, a tablet such as an iPad™), a personal digital assistant (PDA), an Internet appliance, a wearable device such as glasses, or any other type of computing device.

The processor platform 900 of the illustrated example includes the processor 114. The processor 114 of the illustrated example is hardware. For example, the processor 114 can be implemented by one or more integrated circuits, logic circuits, microprocessors or controllers from any desired family or manufacturer. In this example, the processor 114 implements the respiration phase detector 116 and its components (e.g., the example A/D converter 204, the example high-pass filter 206, the example signal partitioner 212, the example feature extractor 216, the example data buffer 218, the example low-pass filter 219, the example peak searcher 222, the example ANN 224, the example classifier 226, the example post-processing engine 230, the example breathing rate analyzer 304, the example trainer 309, the example classification verifier 312, the example breathing interval verifier 314, the example output generator 318).

The processor 114 of the illustrated example includes a local memory 913 (e.g., a cache). The processor 114 of the illustrated example is in communication with a main memory including a volatile memory 914 and a non-volatile memory 916 via a bus 918. The volatile memory 914 may be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 916 may be implemented by flash memory and/or any other desired type of memory device. Access to the main memory 914, 916 is controlled by a memory controller. The data buffer 218 and the database 300 of the respiration phase detector 116 may be implemented by the main memory 414, 416.

The processor platform 900 of the illustrated example also includes an interface circuit 920. The interface circuit 920 may be implemented by any type of interface standard, such as an Ethernet interface, a universal serial bus (USB), and/or a PCI express interface.

In the illustrated example, one or more input devices 922 are connected to the interface circuit 920. The input device(s) 922 permit(s) a user to enter data and commands into the processor 114. The input device(s) can be implemented by, for example, an audio sensor, a microphone, a camera (still or video), a keyboard, a button, a mouse, a touchscreen, a track-pad, a trackball, isopoint and/or a voice recognition system.

One or more output devices 234, 924 are also connected to the interface circuit 920 of the illustrated example. The output devices 234, 924 can be implemented, for example, by display devices (e.g., a light emitting diode (LED), an organic light emitting diode (OLED), a liquid crystal display, a cathode ray tube display (CRT), a touchscreen, a tactile output device, a printer and/or speakers). The interface circuit 920 of the illustrated example, thus, typically includes a graphics driver card, a graphics driver chip or a graphics driver processor.

The interface circuit 920 of the illustrated example also includes a communication device such as a transmitter, a receiver, a transceiver, a modem and/or network interface card to facilitate exchange of data with external machines (e.g., computing devices of any kind) via a network 926 (e.g., an Ethernet connection, a digital subscriber line (DSL), a telephone line, coaxial cable, a cellular telephone system, etc.).

The processor platform 900 of the illustrated example also includes one or more mass storage devices 928 for storing software and/or data. Examples of such mass storage devices 928 include floppy disk drives, hard drive disks, compact disk drives, Blu-ray disk drives, RAID systems, and digital versatile disk (DVD) drives.

The coded instructions 932 of FIG. 8 may be stored in the mass storage device 928, in the volatile memory 914, in the non-volatile memory 916, in the local memory 913, and/or on a removable non-transitory computer readable storage medium such as a CD or DVD.

From the foregoing, it will be appreciated that methods, systems, and apparatus have been disclosed to detect respiration phases (e.g., inhalation and exhalation) based on nasal bridge vibration data collected from a user via, for example, a head-mounted device such as a glasses. Disclosed examples utilize a self-learning artificial neural network (ANN) to detect respiration phases based on one or more features (e.g., energy levels) of the vibration signal data collected from the user. Disclosed examples filter the data to remove noise generated from, for example, movements by the user. Disclosed examples train the ANN using data indicative of a substantially consistent breathing interval such that the ANN to improve efficiency and/or reduce errors with respect to the training of the ANN and the recognition by the ANN of the user's breathing patterns. Disclosed examples post-process the respiration phase classifications by the ANN to verify the classifications, correct any errors if needed, and to determine whether the ANN needs to be re-trained in view of, for examples, changes in the breathing signal data. Thus, disclosed examples intelligently and adaptively detect respiration phases for a user.

Example methods, apparatus, systems, and articles of manufacture to detect respiration phases based on nasal bridge vibration data are disclosed herein. The following is a non-exclusive list of examples disclosed herein. Other examples may be included above. In addition, any of the examples disclosed herein can be considered in whole or in part, and/or modified in other ways.

Example 1 includes an apparatus for analyzing vibration signal data collected from a nasal bridge of a subject via a sensor to reduce errors in training an artificial neural network using the vibration signal data. The apparatus includes a feature extractor to determine feature coefficients of the vibration signal data, the artificial neural network to generate a respiration phase classification for the vibration signal data based on the feature coefficients. The apparatus includes a classification verifier to verify the respiration phase classification and an output generator to generate a respiration phase output based on the verification.

Example 2 includes the apparatus as defined in example 1, further including a breathing rate analyzer to determine a breathing interval for the vibration signal data and compare the breathing interval to a breathing interval variance threshold. The apparatus includes a trainer to train the artificial neural network if the breathing interval satisfies the breathing interval variance threshold.

Example 3 includes the apparatus as defined in example 2, wherein the respiration phase classification includes a first value and a second value and wherein the trainer is to train the artificial neural network if a mean of a first value of at least two respiration phase classifications for the vibration signal data or a mean of the second value of at least two respiration phase classifications for the vibration signal data satisfy a re-training threshold

Example 4 includes the apparatus as defined in examples 1 or 2, wherein the feature coefficients include signal energy for the vibration signal data.

Example 5 includes the apparatus as defined in examples 1 or 2, wherein the respiration phase output is one of inhalation or exhalation.

Example 6 includes the apparatus as defined in claim 1, wherein the respiration phase classification is a first respiration phase classification. The artificial neural network is to generate the first respiration phase classification for a first frame of the vibration signal data and the classification verifier is to verify the first respiration phase classification relative to a second respiration phase classification for a second frame of the vibration signal data.

Example 7 includes the apparatus as defined in example 6, further including a low-pass filter to filter the feature coefficients to generate a frame energy sequence.

Example 8 includes the apparatus as defined in example 7, further including a peak searcher to identify a peak in the vibration data based on the frame energy sequence.

Example 9 includes the apparatus as defined in example 6, wherein the classification verifier is to detect an error if the first respiration phase classification is associated with inhalation and the second respiration phase classification is associated with exhalation. The first frame and the second frame are consecutive frames.

Example 10 includes the apparatus as defined in example 9, wherein an energy of the vibration signal data of the first frame and an energy of the vibration data of the second frame are to satisfy a moving average frame energy threshold.

Example 11 includes the apparatus as defined in any of examples 1, 2, or 6, further including a trainer to train the artificial neural network based on the respiration phase output.

Example 12 includes the apparatus as defined in example 11, further including a data buffer to store the feature coefficients. The trainer is to further train the artificial neural network based on the feature coefficients associated with the respiration phase output.

Example 13 includes the apparatus as defined in example 1, further including a breathing interval verifier to determine if a breathing interval for the vibration signal data meets a breathing interval variance threshold, and wherein if the classification verifier detects an error in the respiration phase classification and the breathing interval verifier determines that the breathing interval meets the breathing interval variance threshold, the classification verifier is to generate an instruction for the artificial neural network to be re-trained.

Example 14 includes the apparatus as defined in example 13, wherein the classification verifier is to correct the respiration phase classification by updating the respiration phase classification with a corrected respiration phase classification. The respiration phase output is to include the corrected respiration phase classification.

Example 15 includes the apparatus as defined in example 13, further including a trainer to train the artificial neural network based on the instruction.

Example 16 includes the apparatus as defined in example 15, wherein if the vibration signal data does not satisfy the breathing interval variance threshold, the trainer is to refrain from training the artificial neural network.

Example 17 includes the apparatus as defined in example 1, further including a signal partitioner to divide the vibration signal data into frames. The artificial neural network is to generate a respective respiration phase classification for each of the frames.

Example 18 includes a method for analyzing vibration signal data collected from a nasal bridge of a subject via a sensor. The method includes determining, by executing an instruction with a processor, feature coefficients of the vibration signal data. The method includes generating, by executing an instruction with the processor, a respiration phase classification for the vibration signal data based on the feature coefficients. The method includes verifying, by executing an instruction with the processor, the respiration phase classification. The method includes generating, by executing an instruction with the processor, a respiration phase output based on the verification.

Example 19 includes the method as defined in example 18, further including determining a breathing interval for the vibration signal data, comparing the breathing interval to a breathing interval variance threshold, and if the breathing interval satisfies the breathing interval variance threshold, training an artificial neural network to generate the respiration phase classification.

Example 20 includes the method as defined in example 19, wherein the respiration phase classification includes a first value and a second value. The method further includes training the artificial neural network if a mean of a first value of at least two respiration phase classifications for the vibration signal data or a mean of the second value of at least two respiration phase classifications for the vibration signal data satisfy a re-training threshold.

Example 21 includes the method as defined in examples 18 or 19, wherein the feature coefficients include signal energy for the vibration signal data.

Example 22 includes the method as defined in examples 18 or 19, wherein the respiration phase output is one of inhalation or exhalation.

Example 23 includes the method as defined in example 18, wherein the respiration phase classification is a first respiration phase classification, and further including generating the first respiration phase classification for a first frame of the vibration signal data and verifying the first respiration phase classification relative to a second respiration phase classification for a second frame of the vibration signal data.

Example 24 includes the method as defined in example 23, further including filtering the feature coefficients to generate a frame energy sequence.

Example 25 includes the method as defined in example 24, further including identifying a peak in the vibration data based on the frame energy sequence.

Example 26 includes the method as defined in example 23, further including detecting an error if the first respiration phase classification is associated with inhalation and the second respiration phase classification is associated with exhalation. The first frame and the second frame are consecutive frames.

Example 27 includes the method as defined in example 26, wherein an energy of the vibration signal data of the first frame and an energy of the vibration data of the second frame are to satisfy a moving average frame energy threshold.

Example 28 includes the method as defined in any of examples 18, 19, or 23, further including training an artificial neural network based on the respiration phase output.

Example 29 includes the method as defined in example 18, further including determining if a breathing interval for the vibration signal data meets a breathing interval variance threshold and generating an instruction for an artificial neural network to be trained if an error is detected in the respiration phase classification and if the breathing interval meets the breathing interval variance threshold.

Example 30 includes the method as defined in example 29, further including correcting the respiration phase classification by updating the respiration phase classification with a correction reparation phase classification. The respiration phase output is to include the corrected respiration phase classification.

Example 31 includes the method as defined in example 29, further including training the artificial neural network based on the instruction.

Example 32 includes the method as defined in example 18, further including dividing the vibration signal data into frames and generating a respective respiration phase classification for each of the frames.

Example 33 includes a computer readable storage medium comprising instructions that, when executed, cause a machine to at least determine feature coefficients of vibration signal data collected from a nasal bridge of a subject via a sensor, generate a respiration phase classification for the vibration signal data based on the feature coefficients, verify the respiration phase classification, and generate a respiration phase output based on the verification.

Example 34 includes the computer readable storage medium as defined in example 33, wherein the instructions, when executed, further cause the machine to determine a breathing interval for the vibration signal data, compare the breathing interval to a breathing interval variance threshold, and if the breathing interval satisfies the breathing interval variance threshold, learn to generate the respiration phase classification.

Example 35 includes the computer readable storage medium as defined in example 34, wherein the respiration phase classification includes a first value and a second value and wherein the instructions, when executed, further cause the machine to learn to generate the respiration phase classification if a mean of the first value of at least two respiration phase classifications for the vibration signal data or a mean of the second value of at least two respiration phase classifications for the vibration signal data satisfy a re-training threshold.

Example 36 includes the computer readable storage medium as defined in examples 33 or 34, wherein the feature coefficients include energy coefficients for the vibration signal data.

Example 37 includes the computer readable storage medium as defined in examples 33 or 34, wherein the respiration phase output is one of inhalation or exhalation.

Example 38 includes the computer readable storage medium as defined in example 33, wherein the instructions, when executed, further cause the machine to generate the first respiration phase classification for a first frame of the vibration signal data and verify the first respiration phase classification relative to a second respiration phase classification for a second frame of the vibration signal data.

Example 39 includes the computer readable storage medium as defined in example 38, wherein the instructions, when executed, further cause the machine to filter the feature coefficients to generate a frame energy sequence.

Example 40 includes the computer readable storage medium as defined in example 39, wherein the instructions, when executed, further cause the machine to identify a peak in the vibration data based on the frame energy sequence.

Example 41 includes the computer readable storage medium as defined in example 38, wherein the instructions, when executed, further cause the machine to detect an error if the first respiration phase classification is associated with inhalation and the second respiration phase classification is associated with exhalation. The first frame and the second frame are consecutive.

Example 42 includes the computer readable storage medium as defined in example 41, wherein an energy of the vibration signal data of the first frame and an energy of the vibration data of the second frame are to satisfy a moving average frame energy threshold.

Example 43 includes the computer readable storage medium as defined in any of examples 33, 34, or 38, wherein the instructions, when executed, further cause the machine to learn to generate the respiration phase classification based on the respiration phase output.

Example 44 includes the computer readable storage medium as defined in example 33, wherein the instructions, when executed, further cause the machine to determine if a breathing interval for the vibration signal data meets a breathing interval variance threshold, detect an error in the respiration phase classification, and learn to generate the respiration phase classification if the error is detected and if the breathing interval meets the breathing interval variance threshold.

Example 45 includes the computer readable storage medium as defined in example 44, wherein the instructions, when executed, further cause the machine to correct the respiration phase classification by updating the respiration phase classification with a corrected respiration phase classification, the respiration phase output to include the corrected respiration phase classification.

Example 46 includes the computer readable storage medium as defined in example 33, wherein the instructions, when executed, further cause the machine to divide the vibration signal data into frames and generate a respective respiration phase classification for each of the frames.

Example 47 includes an apparatus including means for identifying a first respiration phase in first nasal bridge vibration data, means for training the means for identifying to identify the first respiration phase in the first nasal bridge vibration data, and means for verifying the first respiration phase identified by the means for identifying. The means for training is to train the means for identifying based on a verification of the first respiration phase by the means for verifying, the means for identifying to identify a second respiration phase in second nasal bridge vibration data based on the training and the verification.

Example 48 includes the apparatus as defined in example 47, wherein the means for identifying includes an artificial neural network.

Example 49 includes an apparatus including means for determining feature coefficients of the vibration signal data, means for generating a respiration phase classification for the vibration signal data based on the feature coefficients, means for verifying the respiration phase classification, and means for generating a respiration phase output based on the verification.

Example 50 includes the apparatus as defined in example 49, wherein the means for generating the respiration phase classification includes an artificial neural network.

Although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent.

What is claimed is:

1. An apparatus for analyzing vibration signal data collected from a nasal bridge of a subject via a sensor to reduce errors in training an artificial neural network using the vibration signal data, the apparatus comprising:
a feature extractor to determine feature coefficients of the vibration signal data, the artificial neural network to generate a respiration phase classification for the vibration signal data based on the feature coefficients;
a classification verifier to verify the respiration phase classification; and
an output generator to generate a respiration phase output based on the verification.

2. The apparatus as defined in claim 1, further including:
a breathing rate analyzer to:
determine a breathing interval for the vibration signal data; and
compare the breathing interval to a breathing interval variance threshold; and
a trainer to train the artificial neural network if the breathing interval satisfies the breathing interval variance threshold.

3. The apparatus as defined in claim 2, wherein the respiration phase classification includes a first value and a second value and wherein the trainer is to train the artificial neural network if a mean of a first value of at least two respiration phase classifications for the vibration signal data or a mean of the second value of at least two respiration phase classifications for the vibration signal data satisfy a re-training threshold.

4. The apparatus as defined in claim 1, wherein the respiration phase output is one of inhalation or exhalation.

5. The apparatus as defined in claim 1, wherein the respiration phase classification is a first respiration phase classification, the artificial neural network to generate the first respiration phase classification for a first frame of the vibration signal data and the classification verifier to verify the first respiration phase classification relative to a second respiration phase classification for a second frame of the vibration signal data.

6. The apparatus as defined in claim 5, wherein the classification verifier is to detect an error if the first respiration phase classification is associated with inhalation and the second respiration phase classification is associated with exhalation, the first frame and the second frame being consecutive frames.

7. The apparatus as defined in claim 6, wherein an energy of the vibration signal data of the first frame and an energy of the vibration data of the second frame are to satisfy a moving average frame energy threshold.

8. The apparatus as defined in claim 1, further including a breathing interval verifier to determine if a breathing interval for the vibration signal data meets a breathing interval variance threshold, and wherein if the classification verifier detects an error in the respiration phase classification and the breathing interval verifier determines that the breathing interval meets the breathing interval variance threshold, the classification verifier is to generate an instruction for the artificial neural network to be re-trained.

9. The apparatus as defined in claim 8, wherein the classification verifier is to correct the respiration phase classification by updating the respiration phase classification with a corrected respiration phase classification, the respiration phase output to include the corrected respiration phase classification.

10. A method for analyzing vibration signal data collected from a nasal bridge of a subject via a sensor, the method comprising:
determining, by executing an instruction with a processor, feature coefficients of the vibration signal data;
generating, by executing an instruction with the processor, a respiration phase classification for the vibration signal data based on the feature coefficients;
verifying, by executing an instruction with the processor, the respiration phase classification; and
generating, by executing an instruction with the processor, a respiration phase output based on the verification.

11. The method as defined in claim 10, further including:
determining a breathing interval for the vibration signal data;
comparing the breathing interval to a breathing interval variance threshold; and
if the breathing interval satisfies the breathing interval variance threshold, training an artificial neural network to generate the respiration phase classification.

12. The method as defined in claim 11, wherein the respiration phase classification includes a first value and a second value and further including training the artificial neural network if a mean of a first value of at least two respiration phase classifications for the vibration signal data or a mean of the second value of at least two respiration phase classifications for the vibration signal data satisfy a re-training threshold.

13. The method as defined in claim 10, wherein the respiration phase classification is a first respiration phase classification, and further including:
generating the first respiration phase classification for a first frame of the vibration signal data; and
verifying the first respiration phase classification relative to a second respiration phase classification for a second frame of the vibration signal data.

14. The method as defined in claim 13, further including detecting an error if the first respiration phase classification is associated with inhalation and the second respiration phase classification is associated with exhalation, the first frame and the second frame being consecutive frames.

15. The method as defined in claim 14, wherein an energy of the vibration signal data of the first frame and an energy of the vibration data of the second frame are to satisfy a moving average frame energy threshold.

16. The method as defined in claim 10, further including:
determining if a breathing interval for the vibration signal data meets a breathing interval variance threshold; and
generating an instruction for an artificial neural network to be trained if an error is detected in the respiration phase classification and if the breathing interval meets the breathing interval variance threshold.

17. The method as defined in claim 16, further including correcting the respiration phase classification by updating the respiration phase classification with a correction reparation phase classification, the respiration phase output to include the corrected respiration phase classification.

18. A computer readable storage medium comprising instructions that, when executed, cause a machine to at least:
determine feature coefficients of vibration signal data collected from a nasal bridge of a subject via a sensor:
generate a respiration phase classification for the vibration signal data based on the feature coefficients;
verify the respiration phase classification; and
generate a respiration phase output based on the verification.

19. The computer readable storage medium as defined in claim 18, wherein the instructions, when executed, further cause the machine to:
generate the first respiration phase classification for a first frame of the vibration signal data; and
verify the first respiration phase classification relative to a second respiration phase classification for a second frame of the vibration signal data.

20. The computer readable storage medium as defined in claim 18, wherein the instructions, when executed, further cause the machine to:
divide the vibration signal data into frames; and
generate a respective respiration phase classification for each of the frames.
